(19) United States
(12) Patent Application Publication
(10) Pub. No.: US 2003/0092724 A1
(43) Pub. Date: May 15, 2003

(54) Combination sustained release-immediate release oral dosage forms with an opioid analgesic and a non-opioid analgesic

(76) Inventors: Huaihung Kao, Syosset, NY (US); Yadi Zeng, Fort Lee, NJ (US); Fai Jim, Franklin Square, NY (US)

Correspondence Address: KRAMER LEVIN NAFTALIS & FRANKEL LLP, 919 THIRD AVENUE, NEW YORK, NY, 10022, US

(21) Appl. No.: 10/245,139

(22) Filed: Sep. 17, 2002

Related U.S. Application Data
(60) Provisional application No. 60/322,667.
(60) Provisional application No. 60/323,546, filed on Sep. 19, 2001.

(51) Int. Cl. A61K 31/485; A61K 9/22

(52) U.S. Cl. 514/282; 424/468

(57) ABSTRACT

The present invention relates to new and useful oral tablet compositions which include an immediate release portion having an opioid analgesic and a non-opioid analgesic, providing for a rapid onset of therapeutic effect, and a sustained release portion of an opioid analgesic and a non-opioid analgesic, providing for a relatively longer duration of therapeutic effect. A multilayer oral dosage form containing a sustained release layer, which includes oxycodone and APAP, hydrocodone and APAP, or oxymorphone and APAP, and an immediate release layer containing the same active ingredients as the sustained release layer, is also disclosed. Also disclosed are oral tablet compositions, containing a sustained release core, which includes oxycodone and APAP, hydrocodone and APAP, or oxymorphone and APAP, and an immediate release coating containing the same active ingredients as the sustained release core, are also disclosed. In addition, methods of making and using such oral tablet compositions are disclosed.

This application claims priority to U.S. Provisional Application Serial No. 60/322,667, filed Sep. 17, 2001 and U.S. Provisional Application Serial No. 60/323,546, filed Sep. 19, 2001, the specifications of which are incorporated by reference into this application in their entirety.

### FIELD OF THE INVENTION

The present invention relates to new and useful oral tablet compositions that include an immediate release portion having a combination of an opioid analgesic and a non-opioid analgesic, which will provide a rapid onset of therapeutic effect, and a sustained release portion with a combination of an opioid analgesic and a non-opioid analgesic, which will provide for a longer duration of therapeutic effect.

### BACKGROUND OF THE INVENTION

Sustained release oral dosage forms of therapeutically active substances are well known in the pharmaceutical arts. These dosage forms provide a relatively long duration of action as the active agent is gradually released in the gastrointestinal tract. However, they may not provide a sufficiently early onset of therapeutic effect as is commonly seen with some immediate release dosage forms. On the other hand, though immediate release dosage forms may provide early onset of activities, the duration of therapeutic effect may be relatively short, which might require repeated dosing every few hours or so. Unless the dosing of the immediate release dosage form is carefully monitored, maintaining a constant plasma level of active agents without wide fluctuations is often difficult. Hence, an orally administered tablet formulation which provides a favorable dissolution profile which may lead to a relatively constant plasma level of active agents and which provides both an early onset and a long duration of therapeutic effect would be highly desirable.

### OBJECTS AND SUMMARY OF THE INVENTION

It is accordingly an object of the present invention to provide a tablet with an opioid analgesic and a non-opioid analgesic which allows both an early onset and a long duration of therapeutic effect. Another object of the present invention is to provide an orally administered pharmaceutical dosage form that can achieve relatively constant plasma levels of opioid and non-opioid analgesics.

It is a further object of the present invention to provide a combination oxycodone and acetaminophen (APAP) tablet which allow for both an early onset and a relatively long duration of therapeutic effect. Another object of the present invention is to provide an orally administered pharmaceutical dosage form that can achieve relatively constant plasma levels of oxycodone and APAP.

It is a further object of the present invention to provide a method of making an orally administered pharmaceutical dosage form such that the desired plasma profile may be achieved by the dosage form. Such a method may be used to provide dosage forms that exhibit an early onset and a relatively long duration of therapeutic effects. Such a method may be used to provide dosage forms that include a combination of an opioid analgesic and a non-opioid analgesic. In addition, such a method may be used to provide dosage forms that include a combination of oxycodone and APAP.

A further object of the present invention is to provide a multilayer tablet which contains active agents in one layer of an opioid analgesic and a non-opioid analgesic and another layer of an opioid analgesic and a non-opioid analgesic, along with a sustained release mechanism. Another object of the invention is to provide a multilayer tablet which contains one layer of oxycodone and APAP and another layer of oxycodone and APAP with a suitable sustained release mechanism. Methods of making a multilayer tablet with an opioid analgesic and a non-opioid analgesic and preferably a method of making a multilayer tablet with oxycodone and APAP as well as using such tablets are also disclosed.

A further object of the present invention is to provide a bilayer tablet which contains one layer of an opioid analgesic and a non-opioid analgesic as active agents, and a second layer with an opioid analgesic and a non-opioid analgesic and a suitable sustained release mechanism. Another object of the invention is to provide a bilayer tablet which contains one layer of oxycodone and APAP, and a second layer of oxycodone and APAP and a suitable sustained release mechanism. Methods of making a bilayer tablet with an opioid analgesic and a non-opioid analgesic and preferably a method of making a bilayer tablet with oxycodone and APAP as well as using such tablets are also disclosed.

A further object of the invention is to provide a tablet which contains a layer of sustained release core and an immediate release coating layer. Another object of the invention is to provide a tablet having a sustained release core comprising the active agents oxycodone and APAP and an immediate release coating comprising the active agents oxycodone and APAP. Methods of making tablets with a sustained release core and an immediate release coating, preferably with oxycodone and APAP included in the core and coating layers, as well as using such tablets are disclosed.

### BRIEF DESCRIPTION OF THE DRAWINGS

The following drawing is illustrative of certain embodiments of the present invention and is not intended to limit the scope of the appended claims.

FIG. 1 is a graphical representation of the dissolution profile generated by a 10/650 mg bilayer tablet with oxycodone and APAP in both layers in 0.1N HCl tested using the USP 24 Basket Method at 37° C. and 100 rpm.

FIG. 2 is a graphical representation of the dissolution profile generated by a 10/325 mg coated tablet with oxycodone and APAP in both the sustained release core and the immediate release coating in 0.1N HCl tested using the USP 24 Basket Method at 37° C. and 100 rpm rotating speed.

### DETAILED DESCRIPTION OF THE INVENTION

The present invention provides certain benefits of an immediate release (IR) tablet formulation and a sustained release (SR) tablet formulation in a single orally administered dosage form. The early onset from the IR portion, combined with the extended duration from the SR portion provides for desirable dissolution profiles. Upon administering the dose, the rapid onset provided by the IR portion may be less than about two hours, preferably less than about 1.5 hours and even more preferably less than about 1.0 hour. The duration of effect provided by the SR portion may be from about 8 to about 24 hours.

In one embodiment of the present invention, a multilayer tablet is provided which has an SR layer and an IR layer. In another embodiment of the present invention, a multilayer tablet is provided, which contains an opioid analgesic and/or a non-opioid analgesic in at least two layers. In preferred embodiments, the opioid may be oxycodone, hydromorphone, oxymorphone, dihydrocodeine, codeine, hydrocodone, or morphine, and salts thereof In an especially preferred embodiment, the opioid is oxycodone. In another preferred embodiment, the non-opioid analgesic may be APAP, aspirin, or ibuprofen. In an especially preferred embodiment, the non-opioid analgesic is APAP. Methods of making such tablets is also provided.

The favorable dissolution profiles provided by embodiments of the present invention can provide relatively constant levels of active agents in the circulating blood. Importantly, this constant level allows the blood concentration of the active agents to be maintained in the therapeutic range (i.e., no less than the amount needed to produce desired therapeutic results, but no more than the amount which would cause an unacceptable level of undesirable side effects or safety concerns). Favorable dissolution profiles of the present invention will release between about 30% to about 65% of the active ingredients within about 1 hours and between about 55% and about 85% of the active ingredients within about 4 hours for twice and thrice a day administration. Favorable dissolution profiles of the present invention will release between about 15% to about 45% of the active ingredients within about 1 hours and between about 35% and about 65% of the active ingredients within about 4 hours and no less than about 70% released in about 6 hours for once a day administration. The release rates of the active agents need not be precisely the same relative to each other. Dosage forms according to the present invention having the above-described dissolution profiles provide therapeutic plasma levels from less than about 2 hours to about 24 hours after administration. Accordingly, such dosage forms may be administered as infrequently as once a day. Alternatively, administration b.i.d. or t.i.d. is also possible.

The multilayer oral dosage forms of the present invention preferably release both active agents at a rate so as to avoid problems associated with dose-dumping upon oral administration.

Likewise, the IR/SR coated oral dosage forms of the present invention preferably release both active agents at a rate which avoids problems associated with dose-dumping upon oral administration.

The term “tablet” as used herein includes tablets of any shape, and includes caplets, which are tablets having a capsule shape. The tablets may be coated with a pharmaceutically acceptable nonfunctional coating material or have a pharmaceutically acceptable color added to the composition prior to compression.

The term “multilayer” as used herein includes tablets having more than one layer, preferably having two or three layers, and more preferably having two layers.

An SR/IR multilayer tablet may be produced by blending and then compressing the components into tablets. For example, a dry process for producing tablets according to this invention may comprise the following steps:

I. blending the following ingredients into either the SR or the IR portion of the tablet:

| Ingredient | Parts by Weight |
|---|---|
| Active agent 1 (e.g., acetaminophen) | 15-75% |
| Active agent 2 (e.g., oxycodone HCl) | 0.5-40% |
| Sustained release agent for the SR portion | 5-70% |
| (e.g., methacrylate copolymer) | |
| Disintegrant for the IR portion (e.g., starch | 3-15% |
| 1500) | |
| Binder (e.g., povidone) | 3-25% |
| Lubricant (e.g., stearic acid) | 0.5-10% |

II. compressing either the SR or IR portion; and

III. adding the other portion of the blend onto the already formed tablet and compress to form a multilayer tablet.

Tablets may be compressed by any process that can form multiple layers (e.g., bilayer tablets with an IR layer and an SR layer). Several such processes are well known to those of ordinary skill in the art. As an example, the commercially available Hata Three-Layer or Hata Easy-Clean high-output, double-sided presses can be used to produce bilayer tablets. Presses of these types generally permit first layer filling and tamping, second layer filling and tamping, followed by compressing the entire multilayered tablet.

The present invention also provides tablets having more than two layers. For example, the present invention includes trilayer tablets having an IR layer, an SR layer, and a third, middle layer separating the IR and SR layers.

The preferred multilayer tablets of this invention include a shaped and compressed tablet made by dry granulating the active agents and pharmaceutical excipients with a granulating agent, drying (if necessary or desired) and milling or sieving the resultant granulations, and then blending with excipients and compressing into a tablet layer. An additional layer is then added by following the same steps.

Preferred tablets having an SR core and an IR coating may be made by blending the SR portion and then compressing the components into core tablets. For example, a dry process for producing SR core tablets according to this invention may comprise the following steps:

I. blending the following ingredients into the SR portion of the core tablet:

| Ingredient | Parts by Weight |
|---|---|
| Active agent 1 (e.g., acetaminophen) | 5-75% |
| Active agent 2 (e.g., oxycodone HCl) | 0.5-50% |
| Sustained release agent for the SR portion | 5-70% |
| (e.g., methacrylate copolymer) | |
| Binder (e.g., povidone) | 3-25% |
| Lubricant (e.g., stearic acid) | 0.5-10% |

II. compressing the SR portion to form core tablet; and

III. preparing the IR coating suspension as follows:

| Ingredient | Parts by Weight |
|---|---|
| Active agent 1 (e.g., acetaminophen) | 5-75% |
| Active agent 2 (e.g., oxycodone HCl) | 0.5-50% |
| Opadry Coating Materials | 0.5-10% |
| Water | Remove during coating |

IV. applying the IR coating to the SR core tablets.

Core tablets may be compressed by any means of forming a tablet. The excipients, granulating agents, matrices, binders, fillers, disintegrants, lubricants and optional materials commonly known in the art can also be added into the tablet. Sustained release mechanisms such as acrylic polymers, methacrylate copolymer, hydroxyalkylcellulose, hydroxypropylcellulose, hydroxymethylcellulose and/or other sustained release mechanisms well known to those of ordinary skill in the art may be used in the SR portion of the tablet. In addition, coating materials well known to those of ordinary skill in the art may be used to prepare the IR portion of an IR coated tablet.

### Example 1

Oxycodone/APAP Bilayer Tablet

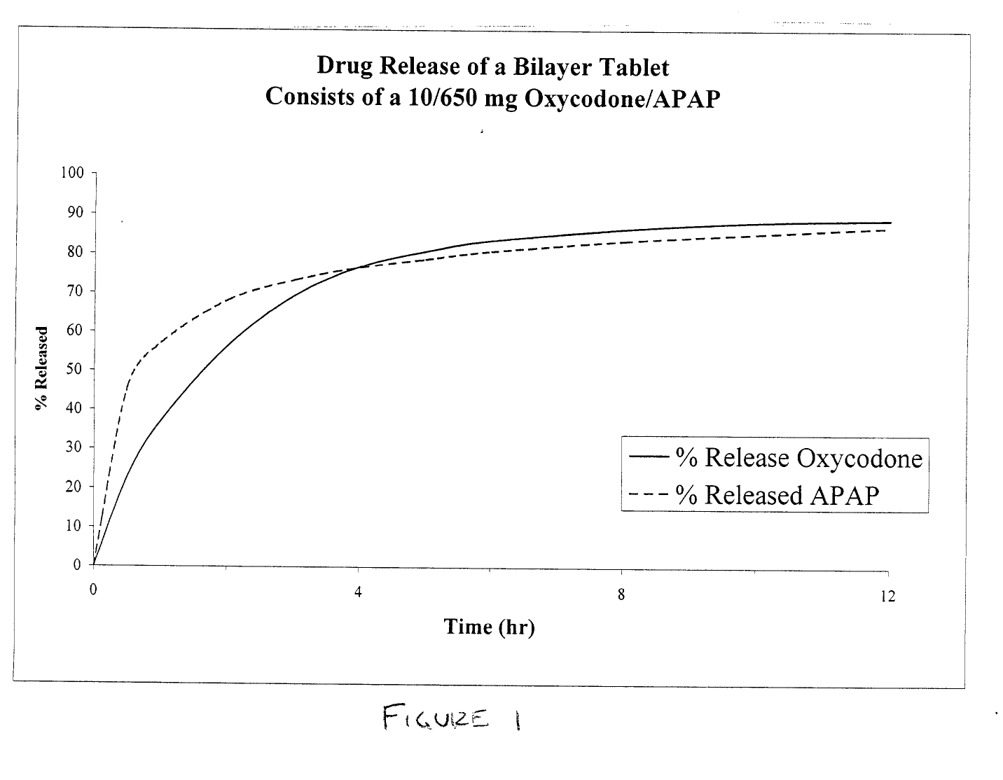

FIG. 1 and Tables 1 and 2 show the data obtained from the dissolution of bilayer tablets with oxycodone/APAP in both layers (10/650 mg) tested using the USP 24 Basket method at 37° C., 100 rpm in 900 ml of 0.1N HCl. The formulation used to produce the dissolution profile in this Example is shown below in Table 3. The profiles indicated a rapid initial dissolution, followed by a prolonged release of both the oxycodone and acetaminophen components.

TABLE 1

| Time | | Percent Oxycodone Dissolved |
|---|---|---|
| 1 | hour | 37 |
| 2 | hours | 56 |
| 4 | hours | 77 |
| 6 | hours | 84 |
| 8 | hours | 86 |
| 10 | hours | 88 |
| 12 | hours | 89 |

TABLE 2

| Time | | Percent Acetaminophen Dissolved |
|---|---|---|
| 1 | hour | 57 |
| 2 | hours | 68 |
| 4 | hours | 77 |
| 6 | hours | 81 |
| 8 | hours | 83 |
| 10 | hours | 85 |
| 12 | hours | 87 |

TABLE 3
Formulation in the Example 1
Oxycodone/APAP 10 mg/650 mg Sustained-Release Tablet

| | mg/tablet |
|---|---|
| IR Layer | |
| Oxycodone hydrochloride | 2.00 |
| Compap-L (90% APAP) | 444.44 |
| Microcrystalline cellulose | 11.00 |
| Magnesium stearate | 2.56 |
| Subtotal | 460.00 |
| SR Layer | |
| Oxycodone hydrochloride | 8.00 |
| APAP | 250.00 |
| Microcrystalline cellulose | 10.00 |
| Eudragit RSPO | 250.00 |
| Sodium lauryl sulfate | 8.00 |
| Povidone | 20.00 |
| Magnesium stearate | 4.00 |
| Subtotal | 550.00 |
| Total Weight per Tablet | 1010.00 |

### Example 2

Oxycodone/APAP IR/SR Tablet

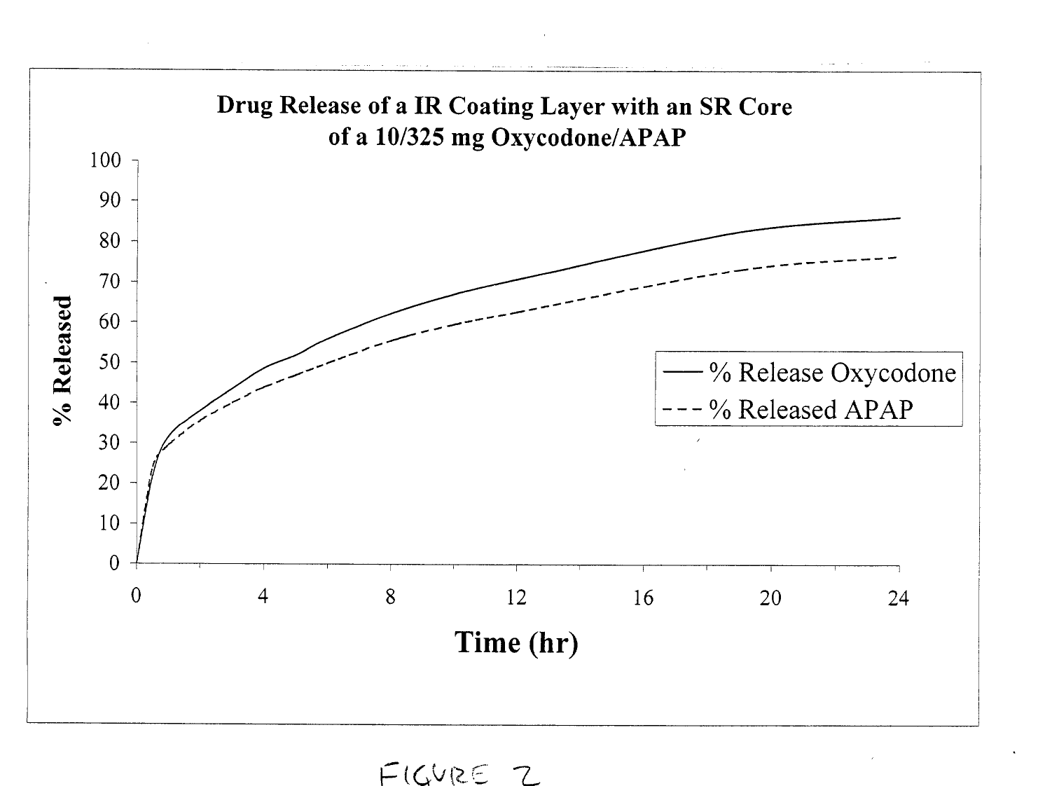

FIG. 2 and Tables 4 and 5 show the data obtained from the dissolution of IR/SR coated tablets with oxycodone/APAP in both the IR and SR portions (10/325 mg) tested using the USP 24 Basket method at 37° C., 100 rpm in 900 ml of 0. IN HCl. The formulation used to produce the dissolution profile in this Example is shown in Table 6. The profiles indicate a rapid initial dissolution, followed by a prolonged release for both the oxycodone and acetaminophen components.

TABLE 4

| Time | | Percent Oxycodone Dissolved |
|---|---|---|
| 1 | hour | 32 |
| 2 | hours | 38 |
| 4 | hours | 49 |
| 6 | hours | 56 |
| 8 | hours | 62 |
| 10 | hours | 67 |
| 12 | hours | 71 |
| 24 | hours | 86 |

TABLE 5

| Time | | Percent Acetaminophen |
|---|---|---|
| 1 | hour | 30 |
| 2 | hours | 36 |
| 4 | hours | 44 |
| 6 | hours | 50 |
| 8 | hours | 56 |
| 10 | hours | 60 |
| 12 | hours | 63 |
| 24 | hours | 77 |

TABLE 6
Formulation in the Example 2
Oxycodone/APAP 10 mg/325 mg Sustained-Release Tablet

| | mg/tablet |
|---|---|
| IR Coating Layer | |
| Oxycodone hydrochloride | 2.00 |
| Compap-L (90% APAP) | 83.33 |
| Opadry Coating | neglient amount |
| Water | remove durign process |
| Subtotal | 85.33 |
| SR Core Tablet Layer | |
| Oxycodone hydrochloride | 8.00 |
| APAP | 305.56 |
| Microcrystalline cellulose | 4.44 |
| Eudragit RSPO | 250.00 |
| Cab-O-Sil | 9.00 |
| Sodium lauryl sulfate | 8.00 |
| Povidone | 20.00 |
| Magnesium stearate | 4.00 |
| Subtotal | 609.00 |
| Total Weight per Tablet | 694.33 |

What is claimed:

1. A multilayer dosage form for oral administration comprising:
at least one layer comprising an opioid analgesic, a non-opioid analgesic, and a sustained release mechanism; and
at least another layer comprising an opioid analgesic and a non-opioid analgesic.

2. The dosage form of claim 1, wherein the dissolution profile for each of the opioid analgesic and non-opioid analgesic is between about 30% and about 65% released in about 1 hour and between about 55% and about 85% released in about 4 hours.

3. The dosage form of claim 2, wherein said dosage form is suitable for twice a day or three times a day administration.

4. The dosage form of claim 1, wherein the dissolution profile for each of the opioid analgesic and non-opioid analgesic is between about 15% and about 45% released in about 1 hour and between about 35% and about 65% released in about 4 hours.

5. The dosage form of claim 4, wherein said dosage from is suitable for once a day administration.

6. The dosage form of any of claims 1-3, wherein the at least one layer comprises oxycodone, APAP, and a sustained release mechanism, and the at least one additional layer comprises oxycodone and APAP.

7. The dosage form of any of claims 1-3, wherein the at least one layer comprises hydrocodone, APAP, and a sustained release mechanism, and the at least one additional layer comprises hydrocodone and APAP.

8. A dosage form of any of claims 1-3, wherein the at least one layer comprises oxymorphone, APAP, and a sustained release mechanism, and the at least one additional layer comprises oxymorphone and APAP.

9. The dosage form of claims 1, 4, or 5, wherein the at least one layer comprises oxycodone, APAP, and a sustained release mechanism, and the at least one additional layer comprises oxycodone and APAP.

10. The dosage form of claims 1, 4, or 5, wherein the at least one layer comprises hydrocodone, APAP, and a sustained release mechanism, and the at least one additional layer comprises hydrocodone and APAP.

11. A dosage form of claims 1, 4, or 5, wherein the at least one layer comprises oxymorphone, APAP, and a sustained release mechanism, and the at least one additional layer comprises oxymorphone and APAP.

12. A method of making a multilayer oral dosage form comprising:
forming at least one layer comprising an opioid analgesic, a non-opioid analgesic, and a sustained release mechanism and compressing it; and,
forming at least one additional layer comprising an opioid analgesic and a non-opioid analgesic and compressing it over the compressed first layer, or vice versa.

13. The method of claim 12, wherein the opioid analgesic is selected from the group consisting of oxycodone, hydrocodone and oxymorphone.

14. The method of claim 12 or 13, wherein the non-opioid analgesic is APAP.

15. A dosage form for oral administration comprising:
a sustained release core layer comprising an opioid analgesic, a non-opioid analgesic, and a sustained release mechanism; and,
a coating layer comprising an opioid analgesic and a non-opioid analgesic.

16. The dosage form of claim 15, wherein the dissolution profile for each of the opioid analgesic and non-opioid analgesic is between about 30% and about 65% released in about 1 hour and between about 55% and about 85% released in about 4 hours.

17. The dosage form of claim 15, wherein said dosage form is suitable for twice a day or three times a day administration.

18. The dosage form of claim 15, wherein the dissolution profile for each of the opioid analgesic and non-opioid analgesic is between about 15% and about 45% released in about 1 hour and between about 35% and about 65% released in about 4 hours.

19. The dosage form of claim 18, wherein said dosage from is suitable for once a day administration.

20. The dosage form of any of claims 15-17, wherein the sustained release core comprises oxycodone, APAP, and a sustained release mechanism, and the IR coating layer comprises oxycodone and APAP.

21. The dosage form of any of claims 15-17, wherein the sustained release core comprises hydrocodone, APAP, and a sustained release mechanism, and the IR coating layer comprises hydrocodone and APAP.

22. The dosage form of any of claims 15-17, wherein the sustained release core comprises oxymorphone, APAP, and a sustained release mechanism, and the IR coating layer comprises oxymorphone and APAP.

23. The dosage form of claims 15, 18, or 19, wherein the sustained release core comprises oxycodone, APAP, and a sustained release mechanism, and the IR coating layer comprises oxycodone and APAP.

24. The dosage form of claims 15, 18, or 19, wherein the sustained release core comprises hydrocodone, APAP, and a sustained release mechanism, and the IR coating layer comprises hydrocodone and APAP.

25. The dosage form of claims 15, 18, or 19, wherein the sustained release core comprises oxymorphone, APAP, and a sustained release mechanism, and the IR coating layer comprises oxymorphone and APAP.

26. A method of making an oral dosage form comprising:
forming a core tablet comprising an opioid analgesic, a non-opioid analgesic, and a sustained release mechanism and compressing it; and,
forming a coating over the core tablet comprising an opioid analgesic and a non opioid analgesic with an immediate release mechanism.

27. The method of claim 26, wherein the opioid analgesic is selected from the group consisting of oxycodone, hydromorphone and oxymorphone.

28. The method of claim 26 or 27, wherein the non-opioid analgesic comprises APAP.

29. A method of treating pain comprising administering the dosage form of any of claims 1-11 and 15-25 to a human or animal.
